(19) United States
(12) Patent Application Publication
(10) Pub. No.: US 2015/0320704 A1
(43) Pub. Date: Nov. 12, 2015

(54) COMPOSITIONS AND METHODS FOR TREATMENT OF HAIR LOSS

(71) Applicant: DS Healthcare Group, Inc., Pompano Beach, FL (US)

(72) Inventor: Daniel Khesin, Boca Raton, FL (US)

(73) Assignee: DS Healthcare Group, Inc., Pompano Beach, FL (US)

(21) Appl. No.: 14/272,920

(22) Filed: May 8, 2014

(51) Int. Cl. A61K 31/19; A61K 31/045

(52) U.S. Cl. CPC A61K 31/19; A61K 31/045

(57) ABSTRACT

Potassium ion channel opening agents, other than ATP-sensitive potassium channel opening agents, have the ability to stimulate hair growth when topically applied to the scalp.

### FIELD OF THE INVENTION

This invention pertains to the field of compositions, methods and formulations for topical treatment of hair loss.

### BACKGROUND OF THE INVENTION

Hair loss, or alopecia, is a common problem with many etiologies that affects hair follicles and is characterized by decreased follicular density and hair shaft diameter, and increased telogen:anagen ratio. Flair loss occurring on the scalp is often progressive, which may lead to partial or full baldness.

Typically, the human scalp contains about 100,000 to 150,000 hairs, of which more than 90% of them are actively growing. Each hair shaft exists for a duration of 3-7 years before being replaced by a new hair. Hair growth on the scalp occurs in a cyclic fashion of three periods: anagen, catagen, and telogen. The anagen phase is the growth phase of the hair shaft and may last for several years. The anagen phase is followed by the catagen phase, which is a short transition stage that signals the end of the active growth of a hair and which lasts for about 2-3 weeks while the hair converts to a club hair. A club hair is formed during the catagen phase when the part of the hair follicle in contact with the lower portion of the hair becomes attached to the hair shaft. This process cuts the hair off from its blood supply and from the cells that produce new hair. When a club hair is completely formed, the hair follicle enters the telogen phase, which is the resting phase of the hair growth cycle and which generally lasts for about 3 months. At the end of the telogen resting phase, the hair falls out and the next anagen phase occurs with rapid cell proliferation in the derma papilla.

Although alopecia of the scalp may have many etiologies, the most common cause of scalp alopecia is male pattern baldness, also known as androgenic alopecia. The etiology of male pattern baldness has not been completely elucidated, although it is known that this condition is related to the hormone dihydrotestosterone (DHT), an analog of the male hormone testosterone. In this condition, the anagen phase of the hair growth cycle is shortened and the telogen phase is extended, resulting in hair thinning and loss. Although this condition is not life threatening or dangerous to health, it leads to social embarrassment and psychological consequences for many of its sufferers.

The United States Food and Drug Administration (FDA) has approved two treatments for male pattern baldness: oral finasteride, which was originally marketed as PROPECIA® by Merck & Co., White House Station, N.J., and topical minoxidil, which was originally marketed as ROGAINE® by Johnson & Johnson, New Brunswick, N.J.

Oral finasteride inhibits the type-II 5α reductase enzyme which is found in the genital, beard, and scalp follicles. This treatment modality works by suppressing DHT and is effective for many men with male patterned baldness at arresting or slowing the course of the condition.

The means by which topical minoxidil functions to stimulate hair growth is discussed in Messenger, British Journal of Dermatology, 150:186-194 (2004). Messenger discloses that the understanding of the mechanism of action of minoxidil on stimulating hair growth is very limited. It is known, however, that minoxidil acts to shorten the telogen phase, which causes premature entry of resting hair follicles into anagen. It may also act to prolong anagen, although this has not been completely elucidated.

Messenger discusses the fact that minoxidil is an agent that causes opening of plasma membrane adenosine triphosphate (ATP)-sensitive potassium channels (K_{ATP} channels), and suggest that the promotion of hair growth by minoxidil is related in some way to this action. In Table 1, on page 188, Messenger points out 3 pieces of evidence in favor of the theory that minoxidil acts on hair growth via potassium channels and 3 pieces of evidence against this theory. Additionally, Messenger discloses that diazoxide and pinacidil, both of which are ATP-sensitive potassium channel opening agents, cause hypertrichosis in humans.

Further, and of more pertinence to the issue of treatment of alopecia, Messenger cites the disclosure of Buhl et al, Journal of Investigative Dermatology, 101:148S-152S (1993) regarding the effect of topical application of minoxidil and three other potassium channel openers on scalp hair growth in balding macaques. In this study, cromakalin and P-1075, each of which is an ATP-sensitive potassium channel opening agent, stimulated hair growth in the macaques over a 20-week treatment period. The fourth agent, RP-49,356, which is likewise an ATP-sensitive potassium channel opening agent, was not effective in promoting hair growth in the macaques.

Tests for determining potassium channel opening agents are well known in the art. Four different types of potassium channels have been identified: (1) Inwardly Rectifiying potassium channels, of which the ATP-sensitive potassium channel is a subset, (2) Calcium-Activated potassium channels, (3) Tandem Pore Domain potassium channels, and (4) Voltage Gated potassium channels. Imaizumi et al, Molecular Pharmacology, 62(4):836-846 (2002), discloses that pimarene compounds are activators of calcium-activated potassium channels. The various potassium channels are differently distributed to cells within the body, and each has a different physiologic function and is activated and blocked by different chemical agents.

To date, several, but not all, ATP-sensitive potassium channel opening chemical agents have been shown to stimulate hair growth when topically applied to the scalp. No other chemical agents, including channel opening agents other than ATP-sensitive potassium channel opening agents, have been shown to have the ability to stimulate hair growth when topically applied to alopecic skin, such as the scalp.

### DETAILED DESCRIPTION OF THE INVENTION

It has been unexpectedly discovered that potassium ion channel opening agents, other than ATP-sensitive potassium channel opening agents, have the ability to stimulate hair growth when topically applied to the scalp.

Accordingly, in a first embodiment this application discloses a formulation for topical application to the scalp in order to stimulate the growth of hair on the areas to which the formulation is applied. The formulation contains a potassium channel opening agent other than an ATP-sensitive potassium channel opening agent in an amount effective to stimulate the growth of hair when topically applied to the scalp.

The potassium channel opening agent may be an inwardly rectifying potassium channel opening agent other than an ATP-sensitive potassium channel opening agent. The inwardly rectifying potassium channel opening agent may be an agent that activates the renal outer medullary potassium (ROMK) channel or may be an agent that activates the G protein coupled inwardly-rectifying (GPCR) potassium channels, such as ifenprodil.

The potassium channel opening agent may be, and preferably is, a calcium-activated potassium channel opening agent, such as a BK (Big Potassium) channel opening agent or an SK (Small Potassium) channel opening agent. Calcium-activated potassium channel opening agents suitable for the present application include BMS-204352, 1-EBIO, NS309, and CyPPA. Additionally, the calcium-activated potassium channel opening agent may be as disclosed as Formula 1 or Formula 2 in Imaizumi, U.S. Pat. No. 7,385,083, the disclosure of which is incorporated in its entirety herein by reference. As a preferred embodiment of the present application, the calcium-activated potassium channel opening agent may be pimaric acid or an analog of pimaric acid, referred to herein as pimarene compounds.

The potassium channel opening agent may be a tandem pore domain potassium channel opening agent. This class of potassium channel opening agents contains many subclasses, including TWIK, TREK, TASK, TALK, and THIK subclasses, each of which is suitable for the present application. An example of a specific tandem pore domain potassium channel opening agent is halothane.

The potassium channel opening agent may be a voltage-gated potassium channel opening agent, such as an hERG or a KvLQT1 voltage-gated potassium channel opening agent. An example of a specific voltage-gated potassium channel opening agent is retigabine.

The formulation for topical application to the scalp may be in any form that is cosmetically acceptable for use on the scalp and which is easily spreadable on the skin of the scalp. Thus, examples of suitable types of formulation include gels, sprays, foams, lotions, creams, and shampoos. The formulation may include, in addition to the potassium channel opening agent, one or more excipients used in cosmetic formulations, such as a solvent or vehicle in which the constituents of the formulation can be dissolved, suspended, disbursed, or emulsified. Such solvent or vehicle is preferably aqueous, alcoholic, or aqueous alcoholic. If the solvent or vehicle contains an alcohol, the alcohol may be one or more of a primary alcohol such as methanol or ethanol, a secondary alcohol such as isopropyl or isobutyl alcohol, or a tertiary alcohol such as tert-butanol. In addition to, or in place of the alcohol, the solvent or vehicle may contain a glycol, such as ethylene, propylene, butylene, or hexylene glycol. The concentration of the alcohol and/or glycol in the formulation is preferably between 1.0% to 95%.

In a preferred embodiment, the solvent or vehicle includes both an alcohol and a glycol, such as ethanol and propylene glycol. If both an alcohol and a glycol are present in the formulation, the ratio of concentration w/w of the alcohol and glycol in the formulation is preferably between 10:1 and 1:10, but may be higher or lower, for example between 99:1 and 1:99. A preferred range of ratios of alcohol and glycol is between 5:1 and 1:5, more preferably between 3:1 and 1:3, and most preferably between 2:1 and 1:2. In one preferred embodiment, the concentration of alcohol, such as ethanol, in the formulation is about 30% and the concentration of glycol, such as propylene glycol, is about 60%.

The formulation may, and preferably does, include additional excipients. Such excipients may include thickening and/or gelling agent, humectants, emollients, pH stabilizing agents, preservatives, antioxidants, wetting agents, stabilizers, lubricants, emulsifiers, colors, fragrances, vitamins, minerals, and peptides. Preferably but not necessarily, the formulation has a pH of less than 8.0, more preferably between 7.0 and 4.0.

The concentration of the potassium ion channel opening agent and the other components of the formulation will vary, depending on the particular components contained in the formulation and the form of the formulation. The concentration of the potassium ion channel opening agent of the formulation is at a concentration that is effective to stimulate hair growth when applied to the scalp. Typically, the concentration of the potassium ion channel opening agent in the formulation will be between 0.0001 to 40% w/w or higher by weight of the formulation. In a preferred embodiment, the concentration of the potassium on channel opening agent in the formulation is between 1% and 25% w/e. In a more preferred embodiment, the concentration of the potassium ion channel opening agent the formulation is between 2% and 15% w/w.

The potassium ion channel opening agent in the formulation may be, as discussed above, an inwardly rectifying potassium channel opening agent other than an ATP-sensitive potassium channel opening agent, a calcium-activated potassium channel opening agent, a tandem pore domain potassium channel opening agent, or a voltage-gated potassium channel opening agent. In a preferred embodiment, the potassium ion channel opening agent is a calcium-activated potassium channel opening agent.

In a preferred embodiment, the calcium-activated potassium channel opening agent is one disclosed in Imaizumi, U.S. Pat. No. 7,385,083. Examples of such calcium-activated potassium channel opening agents are pimaric acid or an analog thereof having the structure shown as Formula A.

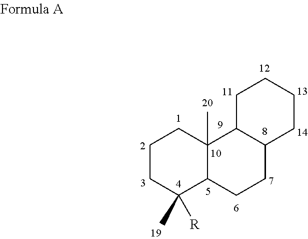

wherein the bond between C7-C8 or C8-C14 is a double bond, R is CHOH or COOH, and 13 is CCH₃C₂H₃ or CCH₃C₂H₅. Possible alternatives for 13 are shown below as alternatives A to D of Formula B.

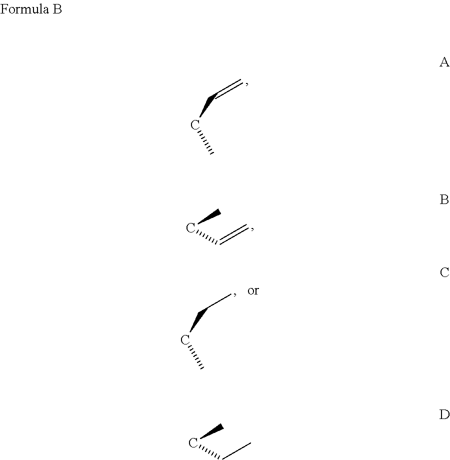

The pimaric acid or analog may further be selected from the group of pharmaceutically acceptable salts and hydrates, biohydrolyzable amides, esters, and imides, and optical isomers, diastereomers, and enantiomers Formula A. At all stereocenters where stereochemistry is not defined, both isomers are included. Preferred stereochemistry at all stereocenters of the compounds mimics that of the naturally occurring pimaric compounds. Examples of methods for synthesis are disclosed in International Published Patent Application Number: EP 2410030A1. Particular pimaric acid analogs that are suitable for the present application include pimaric acid, isopimaric acid, sandaracopirnaric acid, dihydropimaric acid, dihydroisopimaric acid, and dihydmisopimarinol.

In another embodiment, this application discloses a method for making a formulation for topical application to the scalp in order to stimulate the growth of hair. According to this embodiment, a potassium channel opening agent as described above, and which preferably is calcium-activated potassium channel opening agent and most preferably pimaric acid or an analog thereof, is combined with a suitable solvent or vehicle in which the it may be dissolved, suspended, disbursed, or emulsified. If desired, additional components, such as thickening and/or gelling agent, humectants, emollients, pH stabilizing agents, preservatives, antioxidants, wetting agents, stabilizers, lubricants, emulsifiers, colors, fragrances, vitamins, minerals, and peptides may be combined with the pimaric acid or analog in the solvent or vehicle in order to make the formulation.

If the formulation is to be a multi-phase formulation, such as an oil-in-water or a water-in-oil emulsion formulation, the aqueous phase components of the formulation may be combined in a first vessel as an aqueous formulation and the oil phase components of the formulation may be combined in a second vessel, as a separate oil-based formulation, and then the aqueous formulation and the oil-based formulation may be combined into a single vessel and mixed until homogenous to obtain the final formulation.

In another embodiment, this application discloses a method for treatment of alopecia, such as male-pattern baldness. In accordance with this method, the formulation described above is topically applied to alopecic area of skin, such as scalp, in an amount effective to promote the growth of hair in the area to which the formulation is applied. The formulation is applied at a frequency and for a duration sufficient to promote the growth of hair. Preferably, application of the formulation is continued following the regrowth of hair to the alopecic area in order to maintain the continued presence of hair in the area.

Preferably, the formulation is applied daily, such as once or twice daily, although application may be more frequent, such as several times daily, or less frequent, such as 3 to 5 times per week, or every other day, or even once or twice weekly, if such frequency is determined to be sufficient in a particular instance to promote and or maintain the growth of hair to an alopecic area, such as the scalp.

The invention is further disclosed in the following non-limiting examples. In the examples that follow, pimarene compounds such as pimaric acid and analogs are utilized to illustrate the invention. It is understood, however, that the examples are merely exemplary and that the method described herein may be practiced with any potassium channel opening agent.

### Example 1

### Hair Regrowth Study

A double blind study was performed on male subjects between the ages of 25 and 55 years who had been experiencing active thinning-hair. Each of the subjects was evaluated before the study and were found to have otherwise healthy scalps. A global photograph of each subject was taken from the crown area and a second photograph was taken of the top of the head to track frontal areas. Each subject then had to appear daily for a period of 90 days and a technician applied either a test formulation containing two pimarene compounds, pimaric acid and isopimaric acid, in a vehicle of alcohol and glycol (30% ethanol and 62% propylene glycol w/w) or a control formulation containing only vehicle. In addition to the pimarene compounds, the formulations contained pine resin compounds that are not calcium channel opening agents, including abietic acid, neoabietic acid, dehydroabietic acid, and palustric acid. The target sites of each subject were monitored over the 90 day period and photographs were again obtained at the end of this period and compared to those at baseline.

Of the 18 subjects who were treated with the test formulation, only 2 (11%) did not experience a positive response to the study treatment. The treatment resulted in an improvement in hair thickness and growth in sixteen (16) of the 18 subjects (89%). Of these, three (3) of the subjects (17% of the total subjects) experienced a minimal growth in hair, ten (10) of the subjects (55% of subjects) experienced moderate hair growth in the treated areas, and three (17%) experienced a dramatic and dense improvement in hair growth. None of the subjects treated with the control formulation experienced noticeable hair growth in the treated areas.

### Example 2

The treatment study of Example 1 is repeated utilizing a similar formulation containing the vehicle and a pimarene compound selected from the group consisting of pimaric acid, isopimaric acid, sandaracopimaric acid, dihydropimaric acid, dihydroisopimaric acid, and dihydroisopimarinol, and lacking pine resin compounds other than pimarene compounds. The treatment results in an improvement in hair thickness and growth in a majority of the subjects treated with the formulation.

### Example 3

The treatment study of Example 1 is repeated utilizing a similar formulation containing the vehicle and a potassium channel opening agent that is selected from the group consisting of (1) a calcium-activated potassium channel opening agent other than pimaric acid compounds (BIAS-204352), (2) an inwardly rectifying potassium channel opening agent other than an ATP-sensitive potassium channel opening agent (ifenprodil), (3) a tandem pore domain potassium channel opening agent (halothane), and (4) a voltage-gated potassium channel opening agent (retigabine). The formulation lacks a pine resin compound other than a pimarene compound. The treatment results in an improvement in hair thickness and growth in a majority of the subjects treated with the formulation.

Various modifications of the above described invention will be evident to those skilled in the art. It is intended that such modifications are included within the scope of the following claims.

1. A method for promoting the growth of hair comprising topically applying to the skin of a human subject in need thereof a formulation comprising a potassium ion channel opening agent other than an ATP-sensitive potassium channel opening agent in an amount effective to promote the growth of hair.

2. The method of claim 1 wherein the potassium ion channel opening agent is selected from the group consisting of an inwardly rectifying potassium channel opening agent, a calcium-activated potassium channel opening agent, a tandem pore domain potassium channel opening agent, and a voltage-gated potassium channel opening agent.

3. The method of claim 2 wherein the potassium ion channel opening agent is a calcium-activated potassium channel opening agent.

4. The method of claim 3 wherein the calcium-activated potassium channel opening agent is a BK (Big Potassium) channel opening agent.

5. The method of claim 4 wherein the BK channel opening agent is a pimarene compound.

6. The method of claim 5 wherein the pimerene compound has the structure shown as Formula A: 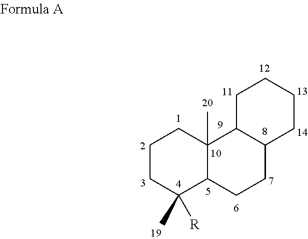
wherein the bond between C₇-C₈ or C₈-C₁₄ is a double bond, R is CHOH or COOH, and position 13 is CCH₃C₂H₃ or CCH₃C₂H₅.

7. The method of claim 7 wherein the structure at position 13 is selected from the group consisting of A to D of Formula B as shown below.

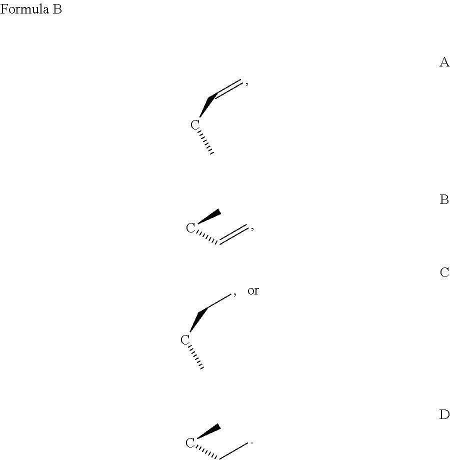

8. The method of claim 5 wherein the pimarene compound is selected from the group consisting of pimaric acid, isopimaric acid, sandaracopimaric acid, dihydropimaric acid, dihydroisopimaric acid, and dihydroisopimarinol.

9. The method of claim 5 wherein the formulation comprises a multiplicity of pimarene compounds.

10. The method of claim 5 wherein the concentration of the pimarene compound in the formulation is between 2% and 15% w/w.

11. The method of claim 1 wherein the skin is on the scalp.

12. The method of claim 1 wherein the formulation is topically applied daily.
